SEQUENCE LISTING (2 sequences)

SEQ ID NO: 1 - DNA, 12 bp, Artificial Sequence
  BUMI tag
gntncncnantn

SEQ ID NO: 2 - DNA, 12 bp, Artificial Sequence
  BUMI tag
nantngngnanc

(19) United States
(12) Patent Application Publication
(10) Pub. No.: US 2015/0072344 A1
(43) Pub. Date: Mar. 12, 2015

(54) Barcoded Universal Marker Indicator (BUMI) Tags

(71) Applicant: Steven R. Wiley, Seattle, WA (US)

(72) Inventor: Steven R. Wiley, Seattle, WA (US)

(73) Assignee: IMDAPTIVE INCORPORATED, Seattle, WA (US)

(21) Appl. No.: 14/481,697

(22) Filed: Sep. 9, 2014

Related U.S. Application Data
(60) Provisional application No. 61/875,851, filed on Sep. 10, 2013.

(51) Int. Cl. C12Q 1/68

(52) U.S. Cl. CPC C12Q 1/6876; C12Q 2600/178
USPC 435/6.11; 536/23.1

(57) ABSTRACT

The BUMI tag is an invention which allows different species of mRNAs from different samples to be quantitatively measured at the first strand cDNA generation step, and is not affected by variations in amplification efficiency of different species of molecules, regardless of amplification method. It consists of a blend of defined nucleotides which comprise the bar-coding portion of the tag along with a set of randomly synthesized nucleotides which comprise the UMI (universal marker indicator) portion of the tag. This blend of Barcode and UMI parts comprises the BUMI tag. The two are interspersed so that the fixed nucleotides of the barcode do not form a contiguous region which might cause biases between different barcodes due to undesired complementarity between the barcode and amplification primers/adaptors.

### CROSS-REFERENCE TO RELATED APPLICATIONS

This application claims all benefits and rights of priority of U.S. application Ser. No. 61/875851, filed on 10 Sep. 2013, the entire disclosure of which is incorporated by reference herein.

### FEDERALLY SPONSORED RESEARCH

None.

### THE NAMES OF THE PARTIES TO A JOINT RESEARCH AGREEMENT

Imdaptive Incorporated and Tria Bioscience Corporation are parties to a joint research agreement.

### SEQUENCE LISTING

Submitted electronically and incorporated by reference herein.

### BACKGROUND

Messenger RNAs are a required intermediate between protein production and genomic DNA in all organisms. The expression of mRNAs from genomic DNA varies depending on cell type and condition. It is of interest in a variety of situations to determine which mRNAs are being expressed in an organism's tissue or fluid sample. The most sensitive and precise of these methods require amplification of specific mRNAs by 1) converting the mRNA to cDNA using reverse-transcriptase, 2) amplifying the cDNA by polymerase chain reaction or thermocycling, cloning into a plasmid or other vector and amplifying in a host organism, or combination of the above, and 3) sequencing the amplified cDNA using massively parallel next generation sequencing (NGS) techniques. NGS is a new type of sequencing technology which uses massively parallel molecular techniques to generate large numbers of individual sequence reads with little starting material and low cost-per-read.

During the amplification step, some species of cDNA molecules will amplify faster than others, and therefore counting the occurrences of a sequence from an NGS run does not correlate with the frequency of the mRNA in the starting sample. This invention describes a method of tagging specific cDNA molecules at the first strand cDNA step so that 1) an accurate measure of the frequency of individual mRNAs in the sample can be determined, 2) samples are barcoded so that multiple samples can be mixed either at the amplification or NGS step and still determine from the sample of origin of the resulting sequences, and 3) the blending of randomly incorporated and single fixed nucleotides in the tags minimize the possibility of biasing the resulting data set due to surreptitious complementarity between the sequences and a given barcode.

### Current NGS Based Sampling Methods:

RNA-seq methods, such as whole transcriptome sequencing, are used to profile the content and distribution of complex samples of mRNAs. Most of these methods require a non-quantitative amplification step like PCR which obscures the starting distribution of particular mRNA species in the starting sample. Amplification can be done by in vitro methods like thermocycling or isothermal amplification, or by in vivo library cloning methods such as plasmid or phage libraries.

Recently Kivioja et al (Nature Methods, January 2012, Vol 9, No 1, 72-74) described a technique whereby a random 10-base sequence, called a unique molecular indicator or UMI, is placed at the upstream (5′) end of the cDNA after the first strand synthesis is performed, during the ‘template switch’ step, creating a RACE-like method of sample amplification preparation. After NGS is performed on the sample, the number of different UMI sequences associated with a species of molecule was shown to more accurately reflect the distribution of that molecule in the starting sample than simply counting the total number of occurrences of the molecule in the sequence data set.

### SUMMARY

The invention is an improved way to both barcode and quantitate mRNAs and other nucleic acids. The BUMI tag contains both barcoding single type nucleotides interspersed with randomly synthesized nucleotides. This tag can be incorporated directly into the first strand cDNA. Use of BUMI tags allows for the multiplex analysis of nucleic acid molecules in complex mixtures, and for the enumeration of first strand cDNA synthesis events.

In certain embodiments, the invention provides a polynucleotide comprising a tag polynucleotide, wherein the nucleotide sequence of the tag polynucleotide consists of a certain number of bases of random sequence and a number m of bases of determined sequence, wherein the maximum number of contigous bases of determined sequence equals m−1; or is a polynucleotide comprising a tag polynucleotide, wherein the tag polynucleotide is produced so that its nucleotide sequence consists of a certain number of bases of random sequence and a number m of bases of determined sequence, wherein the maximum number of contigous bases of determined sequence equals m−1.

Additional embodiments of the invention provide a plurality of polynucleotides each comprising at least one tag polynucleotide, wherein each tag polynucleotide is produced so that its nucleotide sequence consists of a certain number of bases of random sequence and a number m of bases of determined sequence, wherein the maximum number of contigous bases of determined sequence equals m−1; or a plurality of polynucleotides produced by a process comprising: providing a determined nucleotide sequence of length m; producing a plurality of polynucleotides, each comprising a tag polynucleotide having said determined nucleotide sequence interspersed with bases chosen at random from A, C, G, and T or U, such that the maximum number of contiguous bases of said determined nucleotide sequence equals m−1. A polynucleotide selected from one of the above plurality of polynucleotides is a further aspect of the invention.

In the polynucleotides of any of these embodiments, the tag polynucleotide can be chemically synthesized. Further, in the tag polynucleotide, the maximum number of contigous bases of determined sequence is, in some embodiments, selected from the group consisting of: three; two; and one. In certain polynucleotides of the invention, the number of bases of random sequence plus the number of bases of determined sequence equals a number selected from the group consisting of: (a) a number between 2 and 200; (b) a number between 4 and 100; (c) a number between 6 and 50; (d) a number between 8 and 20; (e) 10; (f) 12; and (g) 15. As a particular example, the invention provides a polynucleotide wherein the nucleotide sequence of the tag polynucleotide is GNTNCNCNANTN (SEQ ID NO:1), wherein each ‘N’ can be any base.

The invention also provides a method for analyzing a sample, the method comprising hybridizing a polynucleotide of the invention (comprising a tag polynucleotide) to nucleic acid in the sample. In particular embodiments, the nucleic acid in the sample is mRNA. In additional aspects of the invention, this method further comprises adding a polymerase to the sample. A kit comprising at least one polynucleotide comprising a tag polynucleotide is another embodiment of the invention.

### DESCRIPTION OF DRAWINGS

FIG. 1: The method of construction of an example BUMI tag, with the barcode sequence GTCCAT. The barcode sequence can be selected for a variety of criteria depending on experimental needs. By interspersing the UMI indicator region with the barcode sequence, artifacts arising from spurious sequence homologies to individual barcodes sequences by amplification primers are reduced.

FIG. 2: Incorporation of BUMI tags into DNA sequences during the first strand cDNA synthesis step. After the first stand cDNA is synthesized, amplification by an upstream primer and reverse “landing pad”, primer incorporates the BUMI tag into the sequence, allow normalization of the resulting data set to the number of first strand cDNA events regardless of amplification process.

### DETAILED DESCRIPTION OF THE INVENTION

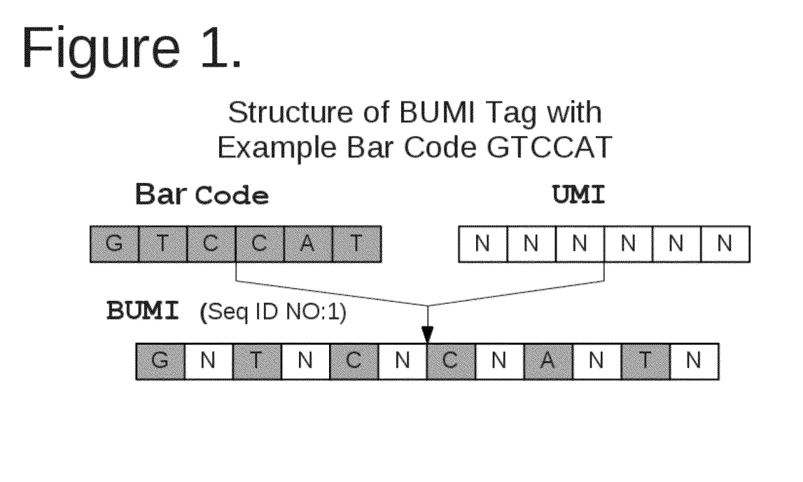

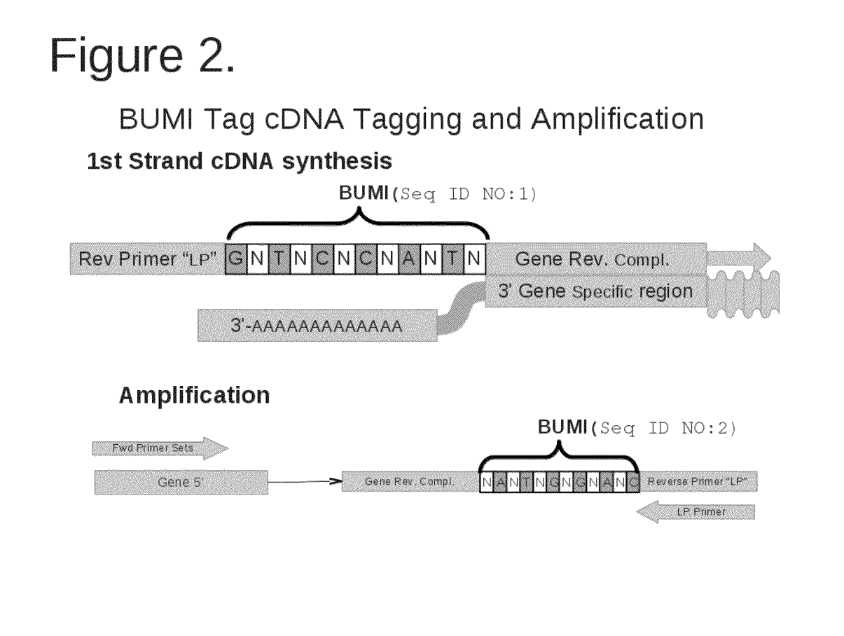

A BUMI tag is a polynucleotide having a multi-nucleotide pattern of fixed nucleotides and randomly synthesized nucleotides. For example, a twelve-base BUMI tag consisting of six fixed and random nucleotide pairs, allows for 4096 different barcodes and 4096 potential nucleotide patterns generated by the random nucleotides (FIG. 1). The tag is placed at the 5′ end of a primer with reverse complementarity to a region at the 3′ end of the mRNA or mRNAs of interest (FIG. 2). Although the figure shows binding to a gene specific region, the primer 3′ end may also anneal to the poly-A tail in other applications of the BUMI tag.

Once the BUMI tag is incorporated into the first strand cDNA the sample may be amplified either by thermocycling amplification, such as PCR, or isothermal amplification, such as LAMP (loop-mediated isothermal amplification), or in-vivo amplification, such as generating a plasmid library, transforming it and growing it in bacteria.

5′ of the BUMI tag is a constant “landing pad” region which provides a location for reverse primer binding during subsequent PCR amplification of the cDNA. The upstream forward primers can be single species or pools of different gene-specific primers complementary to the regions flanking the target sequences, or a primer complementary to a binding site added at the 5′ end of the cDNA by RACE (rapid amplification of cDNA ends) or other 5′ end attachment methods. The landing pad region may also serve as a site to assist in library assembly steps like restriction enzyme generated sticky ends or Gibson assembly, whereby it works as an adaptor for the cloning method.

This procedure incorporates the BUMI tag at the first step before amplification of the sample. Therefore for a given species of mRNA the total number of different sequence patterns in the randomly synthesized portion of the BUMI tag is indicative of the total number of first strand cDNA synthesis events. This allows the user to correct for differences in amplification rates of different messages which arise regardless of amplification technique. These differences in the rate of amplification are a major difficulty when attempting to profile frequencies of low abundance mRNA species in a sample.

Sets of BUMI tags can be chosen so that the barcode portion of each tag in the set has an equal C-G to A-T ratio, and thus eliminates differences in melting temperature between the BUMI tags. The set of BUMI tags may also be selected so that there is a minimum of three nucleotide differences between the barcode portion of each tag, thereby requiring a triple nucleotide sequence substitution before a molecule originating from one BUMI tagged sample becomes mis-identified as originating from a different sample. Longer BUMI tags will allow for a greater minimum required number of substitutions before mis-identification of the origin of one sample for another. An example set of forty five six-nucleotide barcodes that are C-G to A-T balanced and differ from each other by a minimum of three bases is shown in Table 1.

TABLE 1

| Barcode | |
|---|---|
| number | Sequence |
| 1 | ACTCAC |
| 2 | GTCGTA |
| 3 | AGTAGC |
| 4 | ATGCAG |
| 5 | GAGTAG |
| 6 | AGTGAG |
| 7 | TCAGAC |
| 8 | CTCATC |
| 9 | AGCATG |
| 10 | ATACGC |
| 11 | TCATCG |
| 12 | CATCAG |
| 13 | TCACGA |
| 14 | AGAGTC |
| 15 | AGCTGA |
| 16 | ACACTG |
| 17 | CAGTCT |
| 18 | ATAGCG |
| 19 | ACTACG |
| 20 | GCTCTA |
| 21 | TAGCTG |
| 22 | CGAGAT |
| 23 | CACTAC |
| 24 | TGAGCA |
| 25 | TCGCAT |
| 26 | GCTAGT |
| 27 | TGCAGT |
| 28 | GTCTGT |
| 29 | ACGATC |
| 30 | TATGCG |
| 31 | CTGAGT |
| 32 | GATGAC |
| 33 | GTGTCA |
| 34 | ATCGAC |
| 35 | CGTACA |
| 36 | TACGTC |
| 37 | GTGATG |
| 38 | TGACAG |
| 39 | CTGCTA |
| 40 | GAGAGA |
| 41 | TATCGC |
| 42 | ACGTGT |
| 43 | GACACT |
| 44 | TCGACA |
| 45 | TGATGC |

BUMI tags can be ordered from companies that synthesize polynucleotides and oligonucleotides (such as Life Technologies, Carlsbad, Calif.), or chemically synthesized using commercially available machines or by other known methods.

### Variations:

BUMI tags may be of different lengths depending on the required number of barcodes and potential nucleotide patterns generated by the random nucleotides. While the twelve-base BUMI tag (SEQ ID NO:1) shown in FIG. 1 is a reasonable size for most applications, larger or smaller tags can be generated using the same method. BUMI tags can be as short as two bases (one barcode base and one random base), or as long as can be accommodated by the experimental method in which they are used: for example, 100, 200, or more bases in length. Preferably, BUMI tags are between four and 100 bases long; more preferably they are between six and 50 bases long; and most preferably they are between eight and twenty bases long.

Also, the pattern and ratio of fixed (barcode) and random (UMI) bases can be varied, although by avoiding contiguous placement of fixed bases, the probability of the tag having a spurious homology to the 3′ end of amplification primers is reduced. Other examples of the pattern of fixed and random bases include: (a) one fixed base followed by two random bases, with this pattern repeated throughout the length of the BUMI tag; (b) one fixed base followed by one random base, then by one fixed and two random bases, and this unit of five bases repeated for a total of two or more five-base units, followed by a fixed base and then a random base at the end of the BUMI tag; etc. A large number of such variations in the pattern of fixed and random bases can be constructed.

BUMI tags can also incorporate modified nucleotides and nucleotide analogs that are capable of acting as templates for polymerase enzymes, such as methylated nucleotides, biotinylated nucleotides (for example, biotin-11-dUTP or 5-(bio-AC-AP3)dCTP), nucleotides modified with dyes or haptens, boron-modified nucleotides (2′-deoxynucleoside 5′-alpha-[P-borano]-triphosphates), ferrocene-labeled analogs of dTTP (for example, 5-(3-ferrocenecarboxamidopropenyl-1) 2′-deoxyuridine 5′-triphosphate (Fc1-dUTP)), among others. Use of modified nucleotides in BUMI tags can allow PCR products incorporating such tags to be detected by differences in electrophoretic mobility, by fluorescence, by antibody binding, and/or by enzymatic activity, in addition to detection using hybridization and/or sequencing methods.

In another variation of the invention the starting material could be genomic DNA rather than mRNA. In this case the BUMI tag would be attached to one of both ends of a genomic fragment either by ligation of BUMI tagged adapters, or a single template copying step using BUMI tagged primers.

In another variation of the invention the BUMI tag could be incorporated at the 5′ end of the cDNA during the second strand synthesis using a primary primer forward with a BUMI tag followed by a secondary outer primer, or incorporated at the 3′ end using a RACE-like process.

In another variation of the invention the BUMI tags are placed at both the 3′ and 5′ ends of the molecule during the first and second strand cDNA synthesis step.

In another variation of the invention BUMI tags are placed in multiple locations in the target set of gene fragments. For example, a process which assembles a cognate heavy and light chain pair of TCR (T cell receptor) and BCR (B cell receptor) in tandem in a synthetic and/or in-vitro amplified construct could place BUMI tags at 5′ and 3′ ends as well as at internal fusion points where synthesized primers/linkers are incorporated.

In another variation of the invention the target molecule species are all messenger RNA species present in the sample and the landing pad sequence and the BUMI tag is incorporated 5′ of an oligo-dT or anchored oligo-dT first strand cDNA primer, or are incorporated 5′ of a random hexamer primer, for use in oligo-dT or in random primed cDNA synthesis, respectively.

In another variation of the invention the target molecule species are specific sets of messenger RNA species, for example transcripts from complex loci that exhibit somatic cell rearrangement and/or somatic hyper mutation. These include immunoglobulin heavy, kappa, and lambda chain loci, as well as T cell receptor alpha and beta chain loci. In this instantiation, the landing pad sequence and BUMI tag are incorporated 5′ of a gene specific region in the first strand cDNA primer for gene-specific cDNA synthesis. A gene-specific forward or set of forward primers complementary to the region flanking the upstream end of the sequencing target is used for PCR-based amplification or addition of adaptors for cloning-based amplification.

### Advantages of the Method

Unlike standard barcoding methods, in addition to keeping the sample sources identifiable after mixing differently barcoded samples, BUMI tags allow for more accurate determination of the frequencies of molecular species in the starting sample even after amplification by either in vivo or in vitro means.

In certain variations of the invention, in contrast to the UMI method described by Kivioja et al (Nature Methods, January 2012, Vol 9, No 1, 72-74), the BUMI tag is incorporated during the first strand DNA synthesis step and not at the ‘turn around’ step of the Kivioja method, and therefore eliminates biases due to different efficiencies of the reaction for different molecule species at that step.

Furthermore, by interspersing the fixed and mixed nucleotides in the BUMI tag, potential differences in amplification between different barcodes is mitigated, since there are no contiguous barcode specific regions in the cDNA. A small fraction of BUMI tags might have a long region of complementarity to a given PCR primer spanning the fixed and mixed nucleotides, but most members of the BUMI tag have other nucleotides in the mixed positions and therefore the majority cannot contain the long complementary region.

What is claimed is:

1. A plurality of polynucleotides each comprising at least one tag polynucleotide, wherein each tag polynucleotide is produced so that its nucleotide sequence consists of a certain number of bases of random sequence and a number m of bases of determined sequence, wherein the maximum number of contigous bases of determined sequence equals m−1.

2. At least one polynucleotide selected from the plurality of polynucleotides of claim 1.

3. The plurality of polynucleotides of claim 1 wherein at least one tag polynucleotide is chemically synthesized.

4. The plurality of polynucleotides of claim 1 wherein, in the nucleotide sequence of the tag polynucleotide, the maximum number of contigous bases of determined sequence is selected from the group consisting of: three; two;
and one.

5. The plurality of polynucleotides of claim 1 wherein, in the nucleotide sequence of the tag polynucleotide, the number of bases of random sequence plus the number of bases of determined sequence equals a number selected from the group consisting of: (a) a number between 2 and 200; (b) a number between 4 and 100; (c) a number between 6 and 50; (d) a number between 8 and 20; (e) 10; (f) 12; and (g) 15.

6. The plurality of polynucleotides of claim 5 wherein, in the nucleotide sequence of the tag polynucleotide, the number of bases of random sequence plus the number of bases of determined sequence equals a number between 8 and 20.

7. The plurality of polynucleotides of claim 1 wherein the nucleotide sequence of at least one tag polynucleotide is GNTNCNCNANTN (SEQ ID NO:1), wherein each ‘N’ can be any base.

8. A kit comprising the plurality of polynucleotides of claim 1.

9. A kit comprising the plurality of polynucleotides of claim 6.

10. A plurality of polynucleotides produced by a process comprising:
providing a determined nucleotide sequence of length m; producing a plurality of polynucleotides, each comprising a tag polynucleotide having said determined nucleotide sequence interspersed with bases chosen at random from A, C, G, and T or U, such that the maximum number of contiguous bases of said determined nucleotide sequence equals m−1.

11. At least one polynucleotide selected from the plurality of polynucleotides of claim 10.

12. The plurality of polynucleotides of claim 10 wherein at least one tag polynucleotide is chemically synthesized.

13. The plurality of polynucleotides of claim 10 wherein, in the nucleotide sequence of the tag polynucleotide, the maximum number of contigous bases of determined sequence is selected from the group consisting of: three; two; and one.

14. The plurality of polynucleotides of claim 10 wherein, in the nucleotide sequence of the tag polynucleotide, the number of bases of random sequence plus the number of bases of determined sequence equals a number selected from the group consisting of: (a) a number between 2 and 200; (b) a number between 4 and 100; (c) a number between 6 and 50; (d) a number between 8 and 20; (e) 10; (f) 12; and (g) 15.

15. The plurality of polynucleotides of claim 14 wherein, in the nucleotide sequence of the tag polynucleotide, the number of bases of random sequence plus the number of bases of determined sequence equals a number between 8 and 20.

16. The plurality of polynucleotides of claim 10 wherein the nucleotide sequence of at least one tag polynucleotide is GNTNCNCNANTN (SEQ ID NO:1), wherein each ‘N’ can be any base.

17. A kit comprising the plurality of polynucleotides of claim 10.

18. A method for analyzing a sample, the method comprising hybridizing a plurality of polynucleotides to nucleic acid in the sample, wherein each of the plurality of polynucleotides comprises at least one tag polynucleotide, wherein each tag polynucleotide is produced so that its nucleotide sequence consists of a certain number of bases of random sequence and a number m of bases of determined sequence, wherein the maximum number of contigous bases of determined sequence equals m−1.

19. The method of claim 18, wherein the nucleic acid in the sample is mRNA.

20. The method of claim 18 further comprising adding a polymerase to the sample.
